(19) United States
(12) Patent Application Publication
(10) Pub. No.: US 2008/0081067 A1
(43) Pub. Date: Apr. 3, 2008

(54) SUSTAINED RELEASE PHARMACEUTICAL COMPOSITIONS OF VENLAFAXINE AND PROCESS FOR PREPARATION THEREOF

(76) Inventors: Gupta Manishkumar, Gujarat (IN); Rajhans Sujay Kamalakar, Gujarat (IN); Patel Hasmukh Mathurbhai, Gujarat (IN)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 11/841,266

(22) Filed: Aug. 20, 2007

(30) Foreign Application Priority Data
Oct. 3, 2006 (IN) 1627/MUM/2006

(51) Int. Cl. A61K 31/137; A61K 9/22; A61K 9/50; A61P 25/22; A61P 25/24

(52) U.S. Cl. 424/452; 424/457; 424/465; 424/468; 514/648

(57) ABSTRACT

The invention relates to sustained release pharmaceutical compositions of venlafaxine, process for preparing such compositions and method of using such compositions. Preferably, it relates to a sustained release pharmaceutical composition of venlafaxine comprising a first sustained release portion and a second sustained release portion wherein the first and the second sustained release portions are mixed in particular proportion in the formulation.

### CROSS-REFERENCE TO RELATED APPLICATION

This application claims priority to Indian Provisional Application Number 1627/MUM/2006, filed on Oct. 3, 2006, the entire disclosure of which is herein incorporated in its entirety.

### FIELD OF THE INVENTION

The invention relates to sustained release pharmaceutical compositions of venlafaxine, process for preparing such compositions and method of using such compositions. Preferably, it relates to a sustained release pharmaceutical composition of venlafaxine comprising a first sustained release portion and a second sustained release portion, wherein the first and the second sustained release portions are mixed in particular proportion in the formulation.

### BACKGROUND OF THE INVENTION

Venlafaxine and the acid addition salts thereof are disclosed in U.S. Pat. No. 4,535,186. Venlafaxine hydrochloride is chemically designated as [1-(2-(dimethylamino)-1-(4-methoxyphenyl)ethyl)cyclohexanol] having the following formula (I):

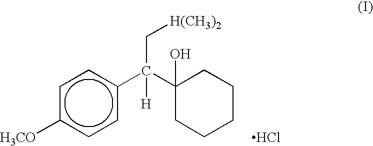

Venlafaxine and its therapeutically acceptable salts have been found to have clinical antidepressant activity by inhibiting monoamine neurotransmitter re-uptake. It is believed that venlafaxine's mechanism of action is related to potent inhibition of the re-uptake of the monoamine neurotransmitters serotonin and norepinephrine and, to a lesser extent, dopamine. However, it has no inhibitory activity on monoamine oxidase.

Venlafaxine hydrochloride is a drug with high water-solubility. In therapeutic dosing with venlafaxine hydrochloride, rapid dissolution results in a rapid increase in blood plasma levels shortly after administration followed by a decrease in blood plasma levels over several hours as the drug is eliminated or metabolized, until sub-therapeutic plasma levels are approached after about twelve hours following administration, thus requiring multiple dosing with the drug. Multiple dosing is inconvenient to patients and may reduce patient compliance. It is desirable to provide an extended or sustained release composition of venlafaxine suitable for once a day dosing.

Extended or sustained release compositions may conventionally be prepared as matrix delivery systems. However, because of the high water-solubility of venlafaxine hydrochloride, matrix delivery system may be ineffective in controlling the rapid initial release of the drug and may not ensure consistent delivery and sustained plasma levels of the drug.

Another alternative for preparing sustained release compositions includes encapsulating the drug and producing sustained release capsules. The WO 99/22724 patent application describes sustained release compositions of venlafaxine in the form of spheroids wherein spheroid core is prepared by extruding and spheronizing a mixture of the drug with microcrystalline cellulose followed by coating with the ethyl cellulose-hydroxypropyl methylcellulose mixture. Commercially available formulation Effexor® XR is supposed to have been formulated using teachings of this patent application for once a day oral administration.

Sustained release compositions may also be provided by multiple unit drug delivery systems comprising beads or pellets wherein an inert core is layered or coated with a drug which may further be layered or coated with a polymer providing sustained release of the drug.

The WO 04/47718 patent application discloses sustained release microbeads containing venlafaxine hydrochloride (up to about 70% w/w) in which venlafaxine hydrochloride is deposited on inert seeds such as sugar spheres using aqueous binder solution to obtain a drug core. The drug core is optionally coated with a layer of non-functional polymer to obtain a hardened drug core. The drug core or hardened drug core is coated with a combination of a functional polymer and a plasticizer to obtain a sustained release composition.

The WO 03/103637 patent application discloses modified release, multiple unit drug delivery systems in which units are prepared by coating the cores with a first layer containing an active pharmaceutical ingredient and a second outer layer comprising a rate controlling polymer. The units optionally contain a seal coat or a film forming layer between core and subsequent layers. The coated units are individually coated with a waxy layer to withstand cracking and to provide favorable mechanical properties. These multiple units are compressed into tablets or filled into capsules or sachets.

The WO 03/41692 patent application discloses extended release composition of venlafaxine hydrochloride wherein venlafaxine hydrochloride is coated on a non-pareil inert core followed by coating of a polymeric layer for controlled release of drug. The process utilizes water, ethanol or its mixture as a solvent for venlafaxine hydrochloride.

The US 2005/106248 patent application discloses a controlled release composition for oral administration comprising an immediate release pellet and an extended release pellet, wherein the immediate release pellet comprises venlafaxine, an inert pellet, and a binder; and the extended release pellet comprises a core of venlafaxine, an inert pellet, a binder, and a coating of water-insoluble polymer surrounding the core.

There remains a need for alternative compositions of venlafaxine which provide sustained release of venlafaxine over the period of about 24 hours, and which also prevents the polymorphic conversion of venlafaxine hydrochloride to other forms. We have surprisingly found that sustained release pharmaceutical composition of venlafaxine can be prepared by providing a first sustained release portion and a second sustained release portion, each portion comprising a core and a functional coat such that at least one of first and second sustained release portion essentially comprises a separating coat or when separating coat is absent, said first or second portion comprises a different weight proportion of functional coat based on the core.

### SUMMARY OF THE INVENTION

In one aspect, the invention provides a sustained release pharmaceutical composition of venlafaxine comprising:

(a) a first sustained release portion comprising one or more units comprising
(i) a core; and
(ii) a functional coat on the core,

(b) a second sustained release portion comprising one or more units comprising
(i) a core;
(ii) a separating coat on the core; and
(iii) a functional coat on the separating coat; and

(c) optionally one or more pharmaceutically acceptable excipients.

In another aspect, the invention provides a sustained release pharmaceutical composition of venlafaxine comprising:

(a) a first sustained release portion comprising one or more units comprising
(i) a core; and
(ii) a functional coat on the core,

(b) a second sustained release portion comprising one or more units comprising
(i) a core;
(ii) a separating coat on the core; and
(iii) a functional coat on the separating coat; and

(c) optionally one or more pharmaceutically acceptable excipients,

wherein the first sustained release portion and the second sustained release portion are present in a ratio ranging from 1 to 9, preferably from 1.5 to 2.5.

In another aspect, the invention provides a sustained release pharmaceutical composition of venlafaxine comprising:

(a) a first sustained release portion comprising one or more units comprising
(i) a core; and
(ii) a functional coat on the core comprising 2 to 15% by weight based on the core,

(b) a second sustained release portion comprising one or more units comprising
(i) a core; and
(ii) a functional coat on the core comprising 15 to 30% by weight based on the core; and

(c) optionally one or more pharmaceutically acceptable excipients.

In another aspect, the invention provides a sustained release pharmaceutical composition of venlafaxine comprising:

(a) a first sustained release portion comprising one or more units comprising
(i) a core; and
(ii) a functional coat on the core comprising 2 to 15% by weight based on the core,

(b) a second sustained release portion comprising one or more units comprising
(i) a core; and
(ii) a functional coat on the core comprising 15 to 30% by weight based on the core; and

(c) optionally one or more pharmaceutically acceptable excipients,

wherein the first sustained release portion and the second sustained release portion are present in a ratio ranging from 0.1 to 1, or from 0.15 to 0.65.

In another aspect, the invention provides a process for preparing a sustained release pharmaceutical composition of venlafaxine, wherein the process comprises:

(a) preparing units of a first sustained release portion comprising:
(i) preparing a core; and
(ii) coating the core with a functional coat,

(b) preparing units of a second sustained release portion comprising:
(i) preparing a core;
(ii) coating the core with a separating coat; and
(iii) coating the product of step (ii) with a functional coat.

(c) mixing the units of the first sustained release portion, the units of the second sustained release portion and optionally one or more pharmaceutically acceptable excipients to obtain the composition.

In another aspect, the invention provides a method for treating major depressive disorder, generalized anxiety disorder and panic disorder; wherein the method comprises administering a patient in need thereof, a sustained release pharmaceutical compositions of venlafaxine.

These and other features, advantages and objects of the present invention will be further understood and appreciated by those skilled in the art by reference to the following specification and claims.

### DETAILED DESCRIPTION OF THE INVENTION

The term “venlafaxine” as used herein may include venlafaxine free base, metabolites of venlafaxine, optically active enantiomer of venlafaxine, or pharmaceutically acceptable acid addition salts thereof or mixtures thereof. It is also intended to include various polymorphic forms of venlafaxine or its pharmaceutically acceptable acid addition salt. The preferred salt of venlafaxine is venlafaxine hydrochloride. A uniform particle size distribution of venlafaxine may be desirable. A preferred particle size distribution of venlafaxine is such that d₅₀ is in the range of 1-100 μm. Venlafaxine may be present in an amount ranging from 10% to 90% by weight of the composition.

The term “sustained release pharmaceutical composition of venlafaxine” as used herein is intended for a composition which provides the desired therapeutic effect of venlafaxine for more than 12 hours, or for a period of 24 hours.

The term “core” as described herein refers to anything which is present below the separating coat or when the separating coat is absent, below the functional coat. When the composition comprises more than one separating coat, the core refers to anything below the separating coat adjacent to the functional coat. For example, the core may comprise any of the non-pareil seed, pellet, bead, granule, mini-tablet, micro-tablet or microcapsule. The non-pareil seeds may be of any pharmaceutically acceptable excipients such as starch, sugar, microcrystalline cellulose, vegetable gums, waxes, and the like. The non-pareil seed may be comprised of starch and sugar. The size of the inert core may vary from 0.1 mm-2 mm. The inert core may be prepared by techniques such as granulation or extrusion-spheronization. For example, the inert core may be prepared by mixing one or more pharmaceutically acceptable excipients, moistening the mixture with water or a solvent, granulating and subsequently drying to obtain granules (inert cores), which granules are coated with venlafaxine to obtain the core. The inert core may also be prepared by mixing one or more pharmaceutically acceptable excipients, wetting with water or organic solvent and mixing in a high shear granulator to form a homogeneous wet mass, extruding the wet mass to form extrudates which are subsequently spheronized to form spheres (inert cores), which spheres are coated with venlafaxine to obtain the core. The core may be present in an amount ranging from 10% to 90% by weight of the composition.

The term “functional coat” as described herein comprises one or more rate-controlling polymers and optionally one or more pharmaceutically acceptable excipients such as plasticizer, anti-tacking agent and opacifying agent.

The term “separating coat” as described herein is present between the core and the functional coat. The separating coat may prevent direct contact of the components of the core and the rate-controlling polymer in the functional coat. The separating coat may also act to modify the sustained release of the drug.

The sustained release composition as described herein comprises units of first sustained release portion and units of second sustained release portion. Each unit of first and second sustained release portion comprises a core, optionally a separating coat on the core and a functional coat on the separating coat or on the core if the separating coat is absent.

The functional coat comprises one or more rate controlling polymers. The rate-controlling polymer may be selected from cellulosic polymers such as ethylcellulose, hydroxypropyl methylcellulose, hydroxypropyl cellulose, methylcellulose, carboxymethylcellulose, hydroxymethylcellulose, and hydroxyethylcellulose; waxes; polyvinylacetate, polymethacrylates such as ammonio methacrylate copolymer, hydrogenated castor oil, and the like. The polymer may be used either alone or in combination with other polymers. Preferably, the rate-controlling polymer is selected from the various pharmaceutically acceptable polymethacrylates such as methacrylic acid co-polymers sold under the brand name EUDRAGIT®. Examples include EUDRAGIT® RS series such as EUDRAGIT® RS 12.5, EUDRAGIT® RS 100, EUDRAGIT® RS PO, EUDRAGIT® RS 30D; and the like. The functional coat may be applied by dispersing or suspending the rate-controlling polymer and optionally a plasticizer, anti-tacking agent and opacifying agent in a suitable medium, such as water or aqueous acidic or alkaline solutions, or in organic solvents such as methanol, ethanol, isopropanol, acetone, methyl ethyl ketone, methylene chloride, ethylene chloride, ethyl acetate, or mixtures thereof, and the resultant solution or suspension may be sprayed directly on the core or separating coat, followed by drying to obtain sustained release units. The functional coat may be present in an amount ranging from 1% to 50% by weight, such as 2-15% or 15-30% by weight based on the core.

The separating coat comprises one or more water soluble polymers, acid soluble polymers, water insoluble polymers; or mixtures thereof. Water-soluble polymer may be selected from hydroxyethyl cellulose, hydroxypropyl cellulose, hydroxypropyl methylcellulose, povidone, polyethylene oxide or mixtures thereof. Acid-soluble polymer may be selected from modified methacrylic acid derivatives or mixtures thereof. Water-insoluble polymer may be selected from ethylcellulose, hydrogenated castor oil, waxes or mixtures thereof. The separating coat may be prepared by dissolving an appropriate amount of the polymer into a suitable solvent system such as water, organic solvent such as alcohol, methylene chloride, and the like; or mixtures thereof, and spraying the solution or suspension on core using a suitable apparatus.

The pharmaceutical compositions as described herein may comprise of one or more pharmaceutically acceptable excipients selected from diluent, disintegrant, binder, surfactant, lubricant, glidant, plasticizer, anti-tacking agent, opacifying agent, and the like.

Diluent may be selected from powdered cellulose, microcrystalline cellulose, silicified microcrystalline cellulose, starch, dibasic calcium phosphate, tribasic calcium phosphate, calcium carbonate, dextrose, kaolin, magnesium carbonate, magnesium oxide; sugars such as lactose or sucrose; sugar alcohols such as mannitol, sorbitol or erythritol; and mixtures thereof. Diluent may be added to increase the bulk volume of the powder to facilitate granulation or compression. The diluent may be present in an amount ranging from 1% to 20% by weight of the composition.

Disintegrant may be selected from croscarmellose sodium, sodium starch glycolate, pregelatinized starch, sodium carboxymethyl cellulose, microcrystalline cellulose, cross-linked polyvinylpyrrolidone and mixtures thereof. The disintegrant may be present in an amount ranging from 1% to 10% by weight of the composition.

Binder may be selected from hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinyl pyrrolidone, pregelatinized starch, sodium alginate, gums, synthetic resins and the like. The binder may be present in an amount ranging from 0.1% to 10% by weight of the composition.

The surfactant may be selected from one or more of non-ionic and ionic (i.e., cationic, anionic and Zwitterionic) surfactants suitable for use in pharmaceutical compositions. Suitable surfactants include mono fatty acid esters of polyoxyethylene sorbitan such as those sold under the brand name Tween®; sodium lauryl sulfate, polyoxyethylene castor oil derivatives such as those sold under the brand name Cremophor®, polyethoxylated fatty acids and their derivatives, propylene glycol fatty acid esters, sterol and sterol derivatives; sorbitan fatty acid esters and their derivatives, sugar esters, polyoxyethylene-polyoxypropylene block copolymers such as those sold under the brand name Poloxamer®, soy lecithin, sodium stearyl fumarate, and the like. The surfactant may be present in an amount ranging from 0.1% to 5% by weight of the composition.

Lubricant, glidant or anti-tacking agent may be selected from talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and mixtures thereof. It would be appreciated that a person skilled in the art is cognizant of the fact that lubricant, glidant or anti-tacking agent may be used interchangeably. The lubricant, glidant or anti-tacking agent may be present in an amount ranging from 0.1% to 20% by weight of the composition.

Plasticizer may be used in a coat to increase the flexibility and strength of the layer and may be selected from propylene glycol, polyethylene glycol, triethyl citrate, acetyl triethyl citrate, diethyl phthalate, dibutyl phthalate, dibutyl sebacate; or mixtures thereof. The plasticizer may be present in an amount ranging from 0.1% to 20% by weight of the composition.

Opacifying agent may be used in a coat to prevent photo-degradation and may be selected from titanium dioxide, iron oxides, and the like. The opacifying agent may be present in an amount ranging from 0.1% to 10% by weight of the composition.

The pharmaceutical compositions as described herein may be prepared by process such as drug-layering. For example, when non-pareil seeds (inert cores) are used, the non-pareil seeds may be coated with a seal coat comprising a film forming polymer, e.g. ethylcellulose, and excipients like plasticizer, anti-tacking agent and opacifying agent. The components of the seal coat may be dissolved or dispersed in an appropriate solvent and the dispersion may be coated on the inert core in a conventional coating pan or fluidized bed equipment (such as a Wurster or Glatt) and the seal-coated non-pareil seeds may then be dried. A coat of venlafaxine may then be applied to such seal-coated inert cores by spraying a suspension or dispersion comprising venlafaxine and excipients, such as binder to obtain the cores. The cores thus obtained may optionally be coated with a separating coat or may directly be coated with the functional coat. The functional coat may be applied by dispersing or suspending the rate-controlling polymer in a suitable medium which may additionally comprise excipients such as plasticizer, anti-tacking agent and opacifying agent, and the resultant dispersion may be sprayed on the cores, followed by drying to obtain sustained release units. The sustained release units may be filled into capsules of suitable size or provided as any suitable composition such as tablet or sachet.

The compositions may also be prepared by providing a core prepared by process such as granulation. For example, pharmaceutically acceptable excipients such as diluent, disintegrant and binder may be mixed; the mixture may be moistened with water or a solvent, granulated and subsequently dried to obtain granules (inert cores) which may be further coated with coat of venlafaxine to obtain the cores. The core may be optionally coated with a separating coat or directly coated with the functional coat by processes as described herein to obtain sustained release units which may be filled into capsules of suitable size or provided as any suitable composition such as tablet or sachet.

The sustained release units obtained as described herein may be subjected to curing. The process of curing involves heating the sustained release units at a temperature of about 40°-70° C. in an apparatus such as an oven or a tray drier. The heating process may be carried out for a period of more than 24 hours. The process of curing helps in minimizing fluctuation in dissolution profiles of sustained release units during storage.

The sustained release pharmaceutical composition of venlafaxine as described herein exhibits a dissolution of not more than 15% in 1 hour, between 30-60% in 4 hours, between 62-80% in 8 hours, between 70-95% in 12 hours, as measured in 900 ml of pH 6.8 phosphate buffer using USP Type II apparatus with a paddle speed of 50 rpm at 37±0.5° C.

In one embodiment, the invention provides the process for preparing the units of the first sustained release portion wherein the process comprises the steps of
preparing an inert core;
coating the inert core with venlafaxine and optionally one or more pharmaceutical acceptable excipients to obtain the core; and
coating the core with one or more rate controlling polymers and optionally one or more pharmaceutically acceptable excipients.

In another embodiment, the invention provides the process for preparing the units of the second sustained release portion wherein the process comprises: preparing an inert core; coating of the inert core with venlafaxine and optionally one or more pharmaceutical acceptable excipients to obtain the core; coating of the core with one or more water soluble polymers and optionally one or more pharmaceutically acceptable excipients; and coating of the polymer coated core with one or more rate controlling polymers and optionally one or more pharmaceutically acceptable excipients.

In another embodiment, units of the first and second sustained release portion may be mixed in a ratio ranging from 1 to 9, or from 1.5 to 2.5, wherein each portion comprises a core and a functional coat such that at least one of first and second sustained release portion essentially comprises a separating coat.

In another embodiment, units of the first and second sustained release portion may be mixed in a ratio ranging from 0.1 to 1, or from 0.15 to 0.65, wherein each portion comprises a core and a functional coat such that first and second portion comprises different weight proportion of functional coat based on the core.

The pharmaceutical compositions as described herein may be illustrated by the following example which is not to be construed as limiting the scope of the invention:

### COMPARATIVE EXAMPLES

| | | Quantity (mg of composition) | |
|---|---|---|---|
| S/N | Ingredients | Example 1 | Example 2 |
| 1) | Non-pareil seeds | 88.07 | 88.07 |

Seal Coat

| | | | |
|---|---|---|---|
| 2) | Ethyl cellulose | 7.93 | 7.93 |
| 3) | Talc | 2.11 | 2.11 |
| 4) | Methanol/Methylene chloride | q.s. | q.s. |

Drug Coat

| | | | |
|---|---|---|---|
| 5) | Venlafaxine hydrochloride | 170 | 170 |
| 6) | Hydroxypropyl methylcellulose | 13.11 | 13.11 |
| 7) | Colloidal silicon dioxide | 13.11 | 13.11 |
| 8) | Methanol/Methylene chloride | q.s. | q.s. |

Separating Coat

| | | | |
|---|---|---|---|
| 9) | Hydroxypropyl methylcellulose | NA | 17.66 |
| 10) | Methanol/Methylene chloride | NA | q.s. |

Functional coat

| | | | |
|---|---|---|---|
| 11) | Ammonio methacrylate copolymer | 33.62 | 89.12 |
| 12) | Dibutyl sebacate | 8.41 | 22.28 |
| 13) | Talc | 16.81 | 44.56 |
| 14) | Acetone/Isopropyl alcohol | q.s. | q.s. |

PROCEDURE: Ethylcellulose and talc were mixed and dispersed in methylene chloride or methanol or a mixture thereof. Non-pareil seeds (inert cores) of appropriate size were coated with the dispersion of ethylcellulose and talc to provide a seal coat. A dispersion of venlafaxine hydrochloride was prepared by mixing venlafaxine hydrochloride, hydroxypropyl methylcellulose and colloidal silicon dioxide and dispersing in methylene chloride or methanol or a mixture thereof. The seal coated non-pareil seeds obtained were coated with the dispersion of venlafaxine hydrochloride to obtain the cores. The cores were coated with a separating coat by spraying a solution of hydroxypropyl methylcellulose in methanol or methylene chloride or mixtures thereof (the separating coat was coated on the cores of Example 2 only). Ammonio methyacrylate copolymer, dibutyl sebacate and talc were dispersed in a mixture of methylene chloride, methanol or a mixture thereof, and the dispersion was sprayed on the separating coat or the cores in the absence of separating coat to obtain sustained release units of venlafaxine hydrochloride.

Dissolution Profiles of Comparative Examples:

% drug dissolved

| Time (in hrs) | Example 1 | Example 2 |
|---|---|---|
| 1 | 2.8 | 0.5 |
| 4 | 44.7 | 0.7 |
| 8 | 99.4 | 14.3 |
| 12 | 103.9 | 42.2 |
| 24 | — | 89.6 |

### Example 3

### Capsules of Venlafaxine Hydrochloride

STEP I (SEAL COATING)

| No. | Ingredients | Qty (kg)/batch |
|---|---|---|
| 1. | Sugar Spheres (25#/30#) | 225.00 |
| 2. | Ethyl Cellulose 10 cps | 20.25 |
| 3. | Talc | 5.40 |
| 4. | Methanol | 171.89 |
| 5. | Methylene Chloride | 286.35 |
| | Total Solid | 250.65 |
| 6. | Talc | 0.5 |

STEP II (DRUG COATING)

| No. | Ingredients | Qty (kg)/batch |
|---|---|---|
| 1. | Seal Coated Sugar Spheres (20#/30#) | 50.0 |
| | (from step I) | |
| 2. | Venlafaxine hydrochloride | 86.64 |
| 3. | Hydroxypropyl methyl cellulose (E15LV) | 6.68 |
| 4. | Colloidal silicon Dioxide (Aerosil 200) | 6.68 |
| 5. | Methanol | 174.24 |
| 6. | Methylene Chloride | 290.40 |
| | Total Solid | 150.00 |
| 7. | Talc | 1.0 |

STEP III (SEPARATING COAT)

| | | Qty (kg)/ |
|---|---|---|
| No. | Ingredients | batch |
| 1. | Drug coated Spheres (from Step II) | 60 |
| 2. | Hydroxypropyl methyl cellulose (6 cps) | 3.6 |
| | (2910) | |
| 3. | Methanol | 24 |
| 4. | Methylene Chloride | 96 |
| | Total Solid | 63.6 |
| 5. | Talc | 0.636 |

STEP IV (POLYMER COATING) (FIRST SUSTAINED
RELEASE PORTION)

| No. | Ingredients | Qty (kg)/batch |
|---|---|---|
| 1. | Drug coated Spheres (from Step II) | 85.00 |
| 2. | Ammonio methacrylate copolymer Type | 9.71 |
| | B (Eudragit RSPO) | |
| 3. | Dibutyl sebacate | 2.43 |
| 4. | Talc | 4.86 |
| 5. | Isopropyl alcohol | 108.24 |
| 6. | Acetone | 108.24 |
| | Total solid content | 102.00 |
| 7. | Talc | 1.28 |

STEP V (POLYMER COATING) (SECOND SUSTAINED
RELEASE PORTION)

| No. | Ingredients | Qty (kg)/batch |
|---|---|---|
| 1. | Spheres having separating coat (from | 62.00 |
| | Step III) | |
| 2. | Ammonio methacrylate copolymer Type B | 17.74 |
| | (Eudragit RSPO) | |
| 3. | Dibutyl sebacate | 4.44 |
| 4. | Talc | 8.87 |
| 5. | Lake of Sunset Yellow | 0.01 |
| 5. | Isopropyl alcohol | 197.64 |
| 6. | Acetone | 197.64 |
| | Total solid content | 93.05 |
| 7. | Talc | 1.40 |

Preparation of Units of First Sustained Release Portion:

Ethylcellulose and talc were mixed and dispersed in methylene chloride or methanol or a mixture thereof. Non-pareil seeds (inert cores) of appropriate size were coated with the dispersion of ethylcellulose and talc to provide a seal coat. A dispersion of venlafaxine hydrochloride was prepared by mixing venlafaxine hydrochloride, hydroxypropyl methylcellulose and colloidal silicon dioxide and dispersing in methylene chloride, methanol or a mixture thereof. The seal coated non-pareil seeds obtained were coated with the dispersion of venlafaxine hydrochloride to obtain the cores. Ammonio methacrylate copolymer, dibutyl sebacate and talc were dispersed in a mixture of methylene chloride, methanol or a mixture thereof, and the dispersion was sprayed on the cores to obtain the sustained release units of venlafaxine. The sustained release units were then optionally cured at 60° C. for 50 hours in oven or tray dryer.

Preparation of Units of Second Sustained Release Portion:

Ammonio methyacrylate copolymer, dibutyl sebacate and talc were dispersed in a mixture of methylene chloride, methanol or a mixture thereof, and the dispersion was sprayed on the separating coat of step III to obtain the sustained release units of venlafaxine. The sustained release units were then optionally cured at 60° C. for 50 hours in oven or tray dryer.

Preparation of the Sustained Release Composition of Venlafaxine:

The units of the first and second sustained release portion were mixed in the ratio of 70:30; the mixture was lubricated and filled into the capsules of appropriate size.

Dissolution Profiles of Example 3:

| Time (in hrs) | % drug dissolved |
|---|---|
| 1 | 3.0 |
| 4 | 50.0 |
| 8 | 71.0 |
| 12 | 81.0 |
| 24 | 100.0 |

### Example 4

### Capsules of Venlafaxine Hydrochloride

STEP I (SEAL COATING)

| No. | Ingredients | Qty (kg)/batch |
|---|---|---|
| 1. | Sugar Spheres (25#/30#) | 225.00 |
| 2. | Ethyl Cellulose 10 cps | 20.25 |
| 3. | Talc | 5.40 |
| 4. | Methanol | 171.89 |
| 5. | Methylene Chloride | 286.35 |
| | Total Solid | 250.65 |

STEP II (DRUG COATING)

| No. | Ingredients | Qty (kg)/batch |
|---|---|---|
| 1. | Seal Coated Sugar Spheres (20#/30#) | 50.0 |
| | (from step I) | |
| 2. | Venlafaxine hydrochloride | 86.64 |
| 3. | Hydroxypropyl methyl cellulose (E15LV) | 6.68 |
| 4. | Colloidal silicon Dioxide (Aerosil 200) | 6.68 |
| 5. | Methanol | 174.24 |
| 6. | Methylene Chloride | 290.40 |
| | Total Solid | 150.00 |
| 7. | Talc | 1.0 |

STEP III (POLYMER COATING)
(FIRST SUSTAINED RELEASE PORTION)

| No. | Ingredients | Qty (kg)/batch |
|---|---|---|
| 1. | Drug coated Spheres (from Step II) | 135.0 |
| 2. | Ammonio methacrylate copolymer | 17.47 |
| | Type B (Eudragit RSPO) | |
| 3. | Dibutyl sebacate | 3.49 |
| 4. | Talc | 8.74 |
| 5. | Isopropyl alcohol | 194.67 |
| 6. | Acetone | 194.67 |
| | Total solid content | 164.70 |
| 7. | Talc | 1.0 |

STEP IV (POLYMER COATING)
(SECOND SUSTAINED RELEASE PORTION)

| No. | Ingredients | Qty (kg)/batch |
|---|---|---|
| 1. | Polymer coated Spheres (from Step III) | 75.00 |
| 2. | Ammonio methacrylate copolymer Type B | 7.94 |
| | (Eudragit RSPO) | |
| 3. | Dibutyl sebacate | 1.59 |
| 4. | Talc | 3.97 |
| 5. | Lake of Sunset Yellow | 0.40 |
| 5. | Isopropyl alcohol | 88.49 |
| 6. | Acetone | 88.49 |
| | Total solid content | 88.90 |
| 7. | Talc | 1.0 |

Preparation of Units of First Sustained Release Portion:

Ethylcellulose and talc were mixed and dispersed in methylene chloride or methanol or a mixture thereof. Non-pareil seeds (inert core) of appropriate size were coated with the dispersion of ethylcellulose and talc to provide a seal coat. A dispersion of venlafaxine hydrochloride was prepared by mixing venlafaxine hydrochloride, hydroxypropyl methylcellulose and colloidal silicon dioxide and dispersing in methylene chloride or methanol or a mixture thereof. The seal coated non-pareil seeds obtained were coated with the dispersion of venlafaxine hydrochloride to obtain the cores. Ammonia methacrylate copolymer, dibutyl sebacate and talc were dispersed in a mixture of methylene chloride or methanol or a mixture thereof, and the dispersion was sprayed on the cores to obtain the units of first sustained release portion.

Preparation of Units of Second Sustained Release Portion:

Units of second sustained release portion were prepared by following the same procedure as for preparing units of first sustained release portion. The cores were coated with a high amount of functional coating.

Preparation of the Sustained Release Composition of Venlafaxine:

The units of the first sustained release portion and the units of the second sustained release portion were mixed in the ratio of 30:70. The mixture was optionally lubricated and filled into the capsules of appropriate size.

Dissolution Profiles of Example 4:

| Time (in hrs.) | % drug dissolved |
|---|---|
| 1 | 5.7 |
| 4 | 35.1 |
| 8 | 66.0 |
| 12 | 78.9 |
| 24 | 89.6 |

TABLE 1
Comparative Dissolution Data of Example 1, 2, 3 and 4 in
USP Type II apparatus, 50 rpm, 900 ml of pH 6.8
phosphate buffer; 37 ± 0.5° C.

| | % drug dissolved | | | | |
|---|---|---|---|---|---|
| | | | Representative | | |
| | Comparative | | examples | | Reference |
| Time | examples | | of the invention | | product |
| (in hrs) | Example 1 | Example 2 | Example 3 | Example 4 | Effexor ® XR |
| 1 | 2.8 | 0.5 | 3.0 | 5.7 | 9 |
| 4 | 44.7 | 0.7 | 50.0 | 35.1 | 40 |
| 8 | 99.4 | 14.3 | 71.0 | 66.0 | 60 |
| 12 | 103.9 | 42.2 | 81.0 | 78.9 | 77 |
| 24 | — | 89.6 | 100.0 | 89.6 | 88 |

### Example 5, 6 and 7

### Capsules of Venlafaxine Hydrochloride

| | Example 5 | | Example 6 | | Example 7 | |
|---|---|---|---|---|---|---|
| | (150 mg) | | (75 mg) | | (37.5 mg) | |
| Ingredients | mg/cap | % w/w | mg/cap | % w/w | mg/cap | % w/w |
| Sugar globules | 88.07 | 21.02 | 44.03 | 21.02 | 22.02 | 18.98 |
| (25#/30#) | | | | | | |
| Ethyl Cellulose | 10.20 | 2.43 | 5.10 | 2.43 | 3.46 | 2.98 |
| (10 cps) | | | | | | |
| Sugar Globules | 25.14 | 6.00 | 12.57 | 6.00 | 16.39 | 14.13 |
| (16#/20#) | | | | | | |
| Venlafaxine | 170.00 | 40.58 | 85.00 | 40.58 | 42.50 | 36.64 |
| hydrochloride | | | | | | |
| Hypromellose | 13.11 | 3.13 | 6.55 | 3.13 | 3.28 | 2.82 |
| E15 LV | | | | | | |
| Colloidal | 13.11 | 3.13 | 6.55 | 3.13 | 3.28 | 2.82 |
| Silicon dioxide | | | | | | |
| (Aerosil 200) | | | | | | |
| Ammonio | 51.68 | 12.33 | 25.84 | 12.33 | 12.92 | 11.14 |
| methacrylate | | | | | | |
| copolymer | | | | | | |
| Type B | | | | | | |
| (Eudragit | | | | | | |
| RSPO) | | | | | | |
| Dibutyl | 12.92 | 3.08 | 6.46 | 3.08 | 3.23 | 2.78 |
| sebacate | | | | | | |
| Talc | 28.54 | 6.81 | 14.27 | 6.81 | 7.38 | 6.36 |
| Hypromellose | 6.18 | 1.48 | 3.09 | 1.48 | 1.55 | 1.33 |
| (6 cps) | | | | | | |
| Lake of sunset | 0.03 | 0.01 | 0.02 | 0.01 | 0.01 | 0.01 |
| yellow | | | | | | |
| Total | 418.97 | 100.00 | 209.48 | 100.00 | 116.00 | 100.00 |

Preparation of Units of First and Second Sustained Release Portion is Similar to that of Example 3

Preparation of the Sustained Release Composition of Venlafaxine:

The units of the first sustained release portion and the units of the second sustained release portion were mixed in the ratio of 65:35. The mixture was optionally lubricated and filled into the capsules of appropriate size.

Dissolution Profile of Example 5: in USP Type II apparatus, 50 rpm, 900 ml of pH 6.8 phosphate buffer; 37±0.5° C.

| Time (in hrs.) | % drug dissolved |
|---|---|
| 1 | 2.45 |
| 4 | 59.35 |
| 8 | 69.35 |
| 12 | 84.9 |
| 24 | 97.9 |

The pharmaceutical compositions as described herein are expected to be bio-equivalent to the reference product, Effexor® XR (sustained release composition of venlafaxine), commercially marketed in the United States.

The above description is considered that of the preferred embodiments only. Modifications of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the doctrine of equivalents.

The invention claimed is:

1. A sustained release pharmaceutical composition of venlafaxine comprising:
(a) a first sustained release portion comprising one or more units comprising
(i) a core; and
(ii) a functional coat on the core,
(b) a second sustained release portion comprising one or more units comprising
(i) a core;
(ii) a separating coat on the core; and
(iii) a functional coat on the separating coat; and
(c) optionally one or more pharmaceutically acceptable excipients.

2. The composition according to claim 1, wherein the core of (a) or (b) comprises venlafaxine free base, metabolites of venlafaxine, optically active enantiomer of venlafaxine, pharmaceutically acceptable acid addition salts thereof or mixtures thereof.

3. The composition according to claim 2, wherein the core is selected from the group consisting of non-pareil seed, pellet, bead, granule, mini-tablet, micro-tablet and microcapsule.

4. The composition according to claim 1, wherein the functional coat of (a) or (b) comprises one or more rate controlling polymers and optionally one or more pharmaceutically acceptable excipients.

5. The composition according to claim 4, wherein the rate controlling polymer is selected from the group consisting of cellulosic polymers, waxes, polyvinylacetate, polymethacrylates and hydrogenated vegetable oils.

6. The composition according to claim 4, wherein the rate controlling polymer is a mixture of ethyl acrylate, methyl methacrylate and trimethylammonioethyl methacrylate.

7. The composition according to claim 1, wherein the separating coat comprises one or more water soluble polymers and optionally one or more pharmaceutically acceptable excipients.

8. The composition according to claim 1, wherein the first sustained release portion and the second sustained release portion are present in a ratio ranging from 1 to 9.

9. The composition according to claim 8, wherein the first sustained release portion and the second sustained release portion are present in a ratio ranging from 1.5 to 2.5.

10. The composition according to claim 1, wherein the composition has a dissolution of not more than 15% in 1 hour, between 30-60% in 4 hours, between 62-80% in 8 hours, between 70-95% in 12 hours, as measured in 900 ml of pH 6.8 phosphate buffer using USP Type II apparatus with a paddle speed of 50 rpm at 37±0.5° C.

11. The composition according to claim 1, wherein one or more pharmaceutically acceptable excipients are selected from the group consisting of diluent, binder, disintegrant, surfactant, plasticizer and glidant.

12. A sustained release pharmaceutical composition of venlafaxine comprising:
(a) a first sustained release portion comprising one or more units comprising
(i) a core; and
(ii) a functional coat on the core comprising 2 to 15% by weight based on the core,
(b) a second sustained release portion comprising one or more units comprising
(i) a core; and
(ii) a functional coat on the core comprising 15 to 30% by weight based on the core; and
(c) optionally one or more pharmaceutically acceptable excipients.

13. The composition according to claim 12, wherein the core of (a) or (b) comprises venlafaxine free base, metabolites of venlafaxine, optically active enantiomer of venlafaxine, pharmaceutically acceptable acid addition salts thereof or mixtures thereof.

14. The composition according to claim 13, wherein the core is selected from the group consisting of non-pareil seed, pellet, bead, granule, mini-tablet, micro-tablet and microcapsule.

15. The composition according to claim 12, wherein the functional coat of (a) or (b) comprises one or more rate controlling polymers and optionally one or more pharmaceutically acceptable excipients.

16. The composition according to claim 15, wherein the rate controlling polymer is selected from the group consisting of cellulosic polymers, waxes, polyvinylacetate, polymethacrylates and hydrogenated vegetable oils.

17. The composition according to claim 15, wherein the rate controlling polymer is a mixture of ethyl acrylate, methyl methacrylate and trimethylammonioethyl methacrylate.

18. The composition according to claim 12, wherein the first sustained release portion and the second sustained release portion are present in a ratio ranging from 0.1 to 1.

19. The composition according to claim 18, wherein the first sustained release portion and the second sustained release portion are present in a ratio ranging from 0.15 to 0.65.

20. The composition according to claim 12, wherein the composition has a dissolution of not more than 15% in 1 hour, between 30-60% in 4 hours, between 62-80% in 8 hours, between 70-95% in 12 hours, as measured in 900 ml of pH 6.8 phosphate buffer using USP Type II apparatus with a paddle speed of 50 rpm at 37±0.5° C.

21. The composition according to claim 12, wherein one or more pharmaceutically acceptable excipients are selected from the group consisting of diluent, binder, disintegrant, surfactant, plasticizer and glidant.

22. The composition according to claim 1 or 12, wherein the composition is in the form of capsule or tablet.

23. A process for preparation of the composition of claim 1 wherein the process comprises:
(a) preparing the units of the first sustained release portion comprising:
(i) preparing a core; and
(ii) coating the core with a functional coat,
(b) preparing the units of the second sustained release portion comprising:
(i) preparing a core;
(ii) coating the core with a separating coat; and
(iii) coating the product of step (ii) with a functional coat,
(c) mixing the units of the first sustained release portion, the units of the second sustained release portion and optionally one or more pharmaceutically acceptable excipients to obtain the composition.

24. The process for preparing the units of the first sustained release portion of claim 23 wherein the process comprises:
(i) preparing an inert core;
(ii) coating the inert core with venlafaxine and optionally one or more pharmaceutical acceptable excipients to obtain the core; and
(iii) coating the core of step (ii) with one or more rate controlling polymers and optionally one or more pharmaceutically acceptable excipients.

25. The process for preparing the units of the second sustained release portion of claim 23 wherein the process comprises:
(i) preparing an inert core;
(ii) coating the inert core with venlafaxine and optionally one or more pharmaceutical acceptable excipients to obtain the core;
(iii) coating the core of step (ii) with one or more water soluble polymers and optionally one or more pharmaceutically acceptable excipients; and
(iv) coating the product of step (iii) with one or more rate controlling polymers and optionally one or more pharmaceutically acceptable excipients.

26. A method for treating major depressive disorder, generalized anxiety disorder and panic disorder, wherein the method comprises administering a patient in need thereof, the composition according to claim 1 or 12.
